(19) United States
(12) Patent Application Publication
(10) Pub. No.: US 2025/0329466 A1
(43) Pub. Date: Oct. 23, 2025

(54) APPLICATION OF LOCAL ANCESTRY INFERENCE AND POLYGENIC RISK SCORES FOR PREDICTION OF COMPLEX DISEASE RISK IN ADMIXED INDIVIDUALS

(71) Applicant: MyOme, Inc., Menlo Park, CA (US)

(72) Inventors: Matthew Rabinowitz, San Francisco, CA (US); Kate M. Im, Ellicott City, MD (US); Tate Tunstall, Menlo Park, CA (US); Dariusz Ratman, Menlo Park, CA (US)

(21) Appl. No.: 19/176,704

(22) Filed: Apr. 11, 2025

Related U.S. Application Data
(63) Continuation of application No. PCT/US23/76737, filed on Oct. 12, 2023.
(60) Provisional application No. 63/379,395, filed on Oct. 13, 2022.

(51) Int. Cl. G16H 50/30; C12Q 1/6869; G16B 20/40; G16B 40/20; G16H 50/70

(52) U.S. Cl. CPC G16H 50/30; C12Q 1/6869; G16B 20/40; G16B 40/20; G16H 50/70

(57) ABSTRACT

Systems, apparatuses, methods, and computer program products are disclosed for generating an admixed PRS for an admixed subject. An example method includes assigning an ancestry label to one or more phased subject genotype segments and generating one or more ancestry specific sets. For each ancestry specific set, the method further includes applying a polygenic risk model to each phased subject genotype segment of the ancestry specific set to generate one or more ancestry specific raw partial PRSs, applying the polygenic risk model to corresponding unadmixed genotype segments to generate one or more unadmixed ancestry raw partial PRSs, determining a mean PRS and a standard deviation PRS for the unadmixed ancestry cohort, normalizing the one or more ancestry specific raw partial PRSs to generate normalized partial PRSs, and generating the admixed PRS for the admixed subject based on a weighted sum of the normalized partial PRSs for each ancestry specific set.

### CROSS-REFERENCE TO RELATED APPLICATION

This application is a continuation of International Application No. PCT/US2023/076737, filed Oct. 12, 2023, which claims the benefit of U.S. Provisional Application No. 63/379,395, filed on Oct. 13, 2022, each of which is incorporated by reference herein in its entirety.

### TECHNOLOGICAL FIELD

The present disclosure relates in general to determining disease risk, and more specifically, to methods for determining a disease occurrence risk for admixed individuals

### BACKGROUND

The present disclosure relates in general to determining disease risk, and more specifically, to methods for determining a disease occurrence risk for admixed individuals

### BRIEF SUMMARY

Polygenic Risk Scores (PRS) have been used to successfully predict complex phenotypes, such as Coronary Artery Disease (CAD) or Breast Cancer (BC). However, their major limitation is lower performance in non-European and recently admixed individuals, which stems from underrepresentation of non-European individuals in publicly available training cohorts.

The proposed method/workflow is meant to improve the performance of PRS models in recently admixed individuals.

The method makes use of multiple PRS scores which demonstrate the best performance for a given ancestry, their effect sizes in unadmixed ancestry individuals and local ancestry decomposition to calculate a single ancestry- and effect-size-weighted PRS score. The obtained composite PRS score can be used as a feature/predictor for a downstream classification model which identifies individuals with elevated disease risk.

Inputs:
Query sample (phased) VCF file
Known ancestry reference (phased) VCF file(s)
PRS model weights for query sample scoring
Effect sizes of PRS models estimated in unadmixed ancestry individuals

Outputs:
Composite PRS score calculated following one of the methods below:
   Sum of partial PRS model scores weighted by global ancestry fraction
   Sum of partial PRS model scores weighted by global ancestry fraction and PRS effect size estimated in unadmixed ancestry individuals
   Sum of partial PRS model scores weighted by global ancestry fraction and partial PRS effect size estimated in unadmixed ancestry individuals

Compared to existing methods using local ancestry deconvolution for PRS our approach includes additional weighting of partial model scores by the effect size of the full or partial PRS model estimated in an independent unadmixed ancestry training cohort while existing methods only weight partial scores by the estimated ancestral fractions and additional scaling factors from other previously used methods.

### BRIEF DESCRIPTION OF THE FIGURES

Having described certain example embodiments in general terms above, reference will now be made to the accompanying drawings, which are not necessarily drawn to scale. Some embodiments may include fewer or more components than those shown in the figures.

FIG. 1 illustrates a schematic block diagram of an example method used to calculate partial ancestry-specific PRS scores and their coefficients using 2-way admixture as an example, in accordance with some example embodiments described herein.

FIG. 2 illustrates performance of the method on a cohort of admixed individuals of Latino or Hispanic origin, in accordance with some example embodiments described herein.

FIG. 3 illustrates a schematic block diagram of example circuitry embodying a device that may perform various operations in accordance with example embodiments described herein.

### DETAILED DESCRIPTION

Some example embodiments will now be described more fully hereinafter with reference to the accompanying figures, in which some, but not necessarily all, embodiments are shown. Because inventions described herein may be embodied in many different forms, the invention should not be limited solely to the embodiments set forth herein; rather, these embodiments are provided so that this disclosure will satisfy applicable legal requirements.

### Definition of Certain Terms

Technical and scientific terms used herein have the meanings commonly understood by one ordinarily skilled in the art to which the present invention pertains, unless otherwise defined. Materials to which reference is made in the following description and examples are obtainable from commercial sources, unless otherwise noted.

The terms “computer-readable medium” and “memory” refer to non-transitory storage hardware, non-transitory storage device or non-transitory computer system memory that may store computer-executable instructions or software programs that may be accessed by a controller, a microcontroller, a computational system or a module of a computational system. A non-transitory computer-readable medium may be accessed by a computational system or a module of a computational system to retrieve and/or execute the computer-executable instructions or software programs stored on the medium. Exemplary non-transitory computer-readable media may include, but are not limited to, one or more types of hardware memory, non-transitory tangible media (for example, one or more magnetic storage disks, one or more optical disks, one or more USB flash drives), computer system memory or random access memory (such as, DRAM, SRAM, EDO RAM), and the like.

The term “computing device” may refer to any computer embodied in hardware, software, firmware, and/or any combination thereof. Non-limiting examples of computing devices include a personal computer, a server, a laptop, a mobile device, a smartphone, a fixed terminal, a personal digital assistant (“PDA”), a kiosk, a custom-hardware device, a wearable device, a smart home device, an Internet-of-Things (“IoT”) enabled device, and a network-linked computing device.

### Example Implementing Apparatuses

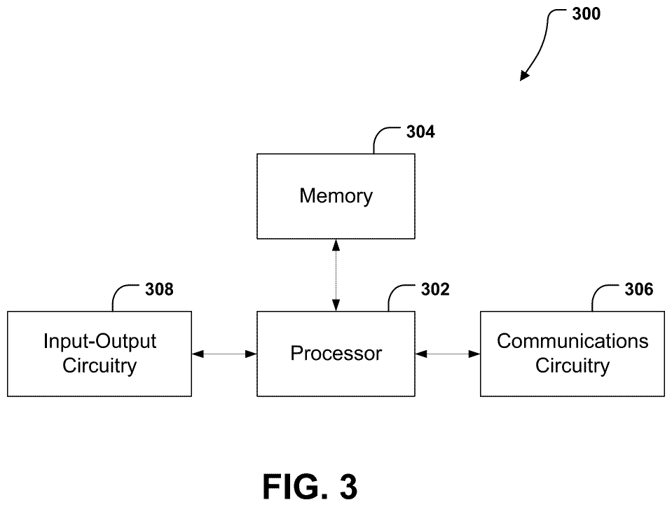

FIG. 3 illustrates an apparatus 300 that may comprise an example system that may implement example embodiments described herein. The apparatus may include processor 302, memory 304, communications circuitry 306, and input-output circuitry 308, each of which will be described in greater detail below, along with any number of additional hardware components not expressly shown in FIG. 3. While the various components are only illustrated in FIG. 3 as being connected with processor 302, it will be understood that the apparatus 300 may further comprise a bus (not expressly shown in FIG. 3) for passing information amongst any combination of the various components of the apparatus 300. The apparatus 300 may be configured to execute various operations described above, as well as those described below in connection with FIG. 3.

The processor 302 (and/or co-processor or any other processor assisting or otherwise associated with the processor) may be in communication with the memory 304 via a bus for passing information amongst components of the apparatus. The processor 302 may be embodied in a number of different ways and may, for example, include one or more processing devices configured to perform independently. Furthermore, the processor may include one or more processors configured in tandem via a bus to enable independent execution of software instructions, pipelining, and/or multithreading. The use of the term “processor” may be understood to include a single core processor, a multi-core processor, multiple processors of the apparatus 300, remote or “cloud” processors, or any combination thereof.

The processor 302 may be configured to execute software instructions stored in the memory 304 or otherwise accessible to the processor (e.g., software instructions stored on a separate storage device). In some cases, the processor may be configured to execute hard-coded functionality. As such, whether configured by hardware or software methods, or by a combination of hardware with software, the processor 302 represent an entity (e.g., physically embodied in circuitry) capable of performing operations according to various embodiments of the present invention while configured accordingly. Alternatively, as another example, when the processor 302 is embodied as an executor of software instructions, the software instructions may specifically configure the processor 302 to perform the algorithms and/or operations described herein when the software instructions are executed.

Memory 304 is non-transitory and may include, for example, one or more volatile and/or non-volatile memories. In other words, for example, the memory 304 may be an electronic storage device (e.g., a computer readable storage medium). The memory 304 may be configured to store information, data, content, applications, software instructions, or the like, for enabling the apparatus to carry out various functions in accordance with example embodiments contemplated herein.

The communications circuitry 306 may be any means such as a device or circuitry embodied in either hardware or a combination of hardware and software that is configured to receive and/or transmit data from/to a network and/or any other device, circuitry, or module in communication with the apparatus 300. In this regard, the communications circuitry 306 may include, for example, a network interface for enabling communications with a wired or wireless communication network. For example, the communications circuitry 306 may include one or more network interface cards, antennas, buses, switches, routers, modems, and supporting hardware and/or software, or any other device suitable for enabling communications via a network. Furthermore, the communications circuitry 306 may include the processing circuitry for causing transmission of such signals to a network or for handling receipt of signals received from a network.

The apparatus 300 may include input-output circuitry 308 configured to provide output to a user and, in some embodiments, to receive an indication of user input. It will be noted that some embodiments will not include input-output circuitry 308, in which case user input may be received via a separate device. The input-output circuitry 308 may comprise a user interface, such as a display, and may further comprise the components that govern use of the user interface, such as a web browser, mobile application, dedicated client device, or the like. In some embodiments, the input-output circuitry 308 may include a keyboard, a mouse, a touch screen, touch areas, soft keys, a microphone, a speaker, and/or other input/output mechanisms. The input-output circuitry 308 may utilize the processor 302 to control one or more functions of one or more of these user interface elements through software instructions (e.g., application software and/or system software, such as firmware) stored on a memory (e.g., memory 304) accessible to the processor 302.

In some embodiments, various components of the apparatus 300 may be hosted remotely (e.g., by one or more cloud servers) and thus not all components must reside in one physical location. Moreover, some of the functionality described herein may be provided by third party circuitry. For example, apparatus 300 may access one or more third party circuitries via any sort of networked connection that facilitates transmission of data and electronic information between the apparatus 300 and the third party circuitries. In turn, the apparatus 300 may be in remote communication with one or more of the components described above as comprising the apparatus 300.

As will be appreciated based on this disclosure, some example embodiments may take the form of a computer program product comprising software instructions stored on at least one non-transitory computer-readable storage medium (e.g., memory 304). Any suitable non-transitory computer-readable storage medium may be utilized in such embodiments, some examples of which are non-transitory hard disks, CD-ROMs, flash memory, optical storage devices, and magnetic storage devices. It should be appreciated, with respect to certain devices embodied by apparatus 300 as described in FIG. 3, that loading the software instructions onto a computing device or apparatus produces a special-purpose machine comprising the means for implementing various functions described herein.

Having described specific components of the apparatus 300, example embodiments are described below.

### Example Operations

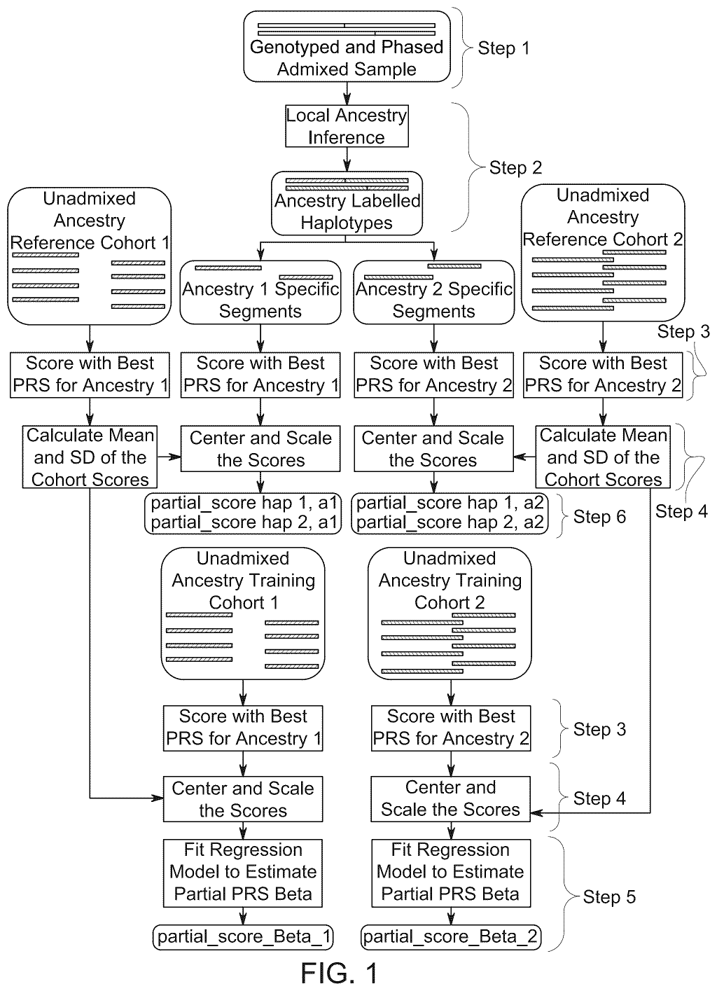

FIG. 1 depicts an example method for calculating partial-ancestry specific PRS scores and their coefficients using 2-way admixture as an example. As noted above, the steps shown in FIG. 1 may be performed by a computing device such as apparatus 300, which is described above.

Step 0. The performance of candidate PRS models for each continental ancestry is evaluated using unadmixed ancestry training cohorts (e.g. UKBB or other cohort with genotypes and phenotype labels available) and the best performing models for each continental ancestry are identified.

Step 1. Patient's DNA sample is collected and subject to Whole Genome Sequencing WGS, genotyping and phasing. This analysis can be accomplished using long-read sequencing techniques (i.e., read lengths of at least about 5 kb or more, including ˜20 kb or more, and ultra long-read sequencing read lengths of about ˜100 kb or more), which services are available by existing vendors such as Pacific Biosciences, Oxford Nanopore Technologies, and Illumina.

Step 2. The local ancestry of a patient sample is estimated using a reference cohort of known ancestry samples such as 1000 Genomes Project and one of the previously described methods.

Following ancestry inference each marker of the patient sample is labeled with its inferred ancestry and haplotypes are partitioned into regions corresponding to each inferred ancestry.

Step 3. Ancestry specific regions of the subject are scored using the best performing PRS model for a given ancestry (as identified in Step 0) to obtain raw partial PRS scores. Simultaneously, the same segments are scored within the unadmixed ancestry reference cohort (such as 1000 Genomes Project samples).

Additionally, in one variation of the method the same regions are scored in unadmixed ancestry individuals of the training cohort for which phenotype information is available (e.g. UKBB or other biobank data).

Step 4. The mean and standard deviation of partial PRS scores in the reference cohort are calculated and used to center and scale each partial PRS score of the patient. Similarly, partial scores of the training cohort are centered and scaled using the same mean and standard deviation.

Step 5. In embodiments of the method which makes use of the unadmixed ancestry training cohort an additional step is performed to estimate the effect size of the ancestry-specific partial PRS score (partial_βᵢ in equation 3) with respect to phenotype of interest. This is accomplished by fitting a linear/logistic regression model for each ancestry with corresponding partial PRS score as a predictor.

An alternative method (not depicted on FIG. 1) to estimate the effect-size of the ancestry specific partial PRS score is to use the effect size of the corresponding full PRS score (βᵢ in equation 2, calculated using complete genomes of training cohort samples). This is also accomplished by fitting a linear/logistic regression.

Step 6. The admixed PRS score for an admixed sample is calculated as a weighted sum of partial PRS scores using one of the 3 equations below:

Equation 1: Composite PRS score with partial scores weighted by global ancestry fractions:

$${score}_{gw} = {\sum }_{i = 1}^{n} ( {partial}_{{score}_{hap 1 , i}} + {partial}_{{score}_{hap 2 , i}} ) * {anc\_fraction}_{i} \qquad ( 1 )$$

Equation 2: Composite PRS score with partial scores weighted by global ancestry fractions and full PRS model effect sizes estimated in independent unadmixed ancestry (training) cohorts.

$${score}_{bw} = {\sum }_{i = 1}^{n} {β}_{i} ( {partial}_{{score}_{hap 1 , i}} + {partial}_{{score}_{hap 2 , i}} ) * {anc\_fraction}_{i} \qquad ( 2 )$$

Equation 3: Composite PRS score with partial scores weighted by global ancestry fractions and partial PRS model effect sizes estimated in independent unadmixed ancestry (training) cohorts.

$${score}_{pbw} = {\sum }_{i = 1}^{n} {partial\_β}_{i} ( {partial}_{{score}_{hap 1 , i}} + {partial}_{{score}_{hap 2 , i}} ) * {anc\_fraction}_{i} \qquad ( 3 )$$

where i indexes fractional ancestry components, partial_score is centered and scaled partial score calculated as described in Step 4, hap1 and hap2 index query sample haplotypes and anc_fraction is a global estimate of the given fractional ancestry (fraction of the genome length assigned to this ancestry).

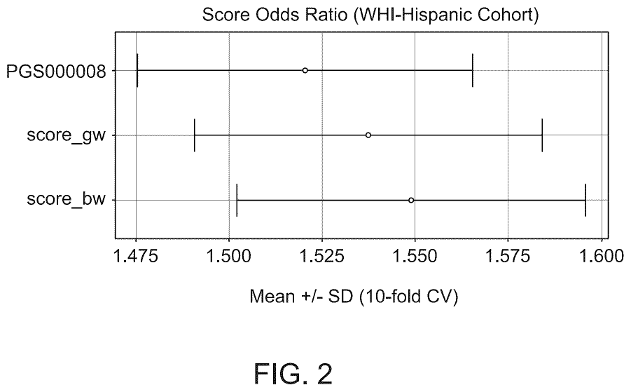

FIG. 2 shows the performance of the method in the cohort of admixed individuals of Latino/Hispanic origin. The PGS000008 is a single PRS model, which does not make use of ancestry inference and is included as a baseline for performance. The score_gw and score_bw are the composite scores calculated following equations 1 and 2, respectively. The value on the x-axis is the odds ratio (expressed in standard deviation units of control samples) from the logistic regression model using breast cancer as an outcome. Error bars correspond to standard deviation of 10× repeated 10-fold cross-validation.

### CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1-15. (canceled)

16. A method for determining an admixed polygenic risk score (PRS) for an admixed subject, the method comprising:
assigning an ancestry label to one or more phased subject genotype segments;
generating one or more ancestry specific groupings, wherein each ancestry specific grouping comprises the one or more phased subject genotype segments corresponding to a particular ancestry label; and
for each ancestry specific grouping:
applying a polygenic risk model corresponding to the ancestry label of the ancestry specific grouping to each phased subject genotype segment of the ancestry specific grouping to generate one or more ancestry specific raw partial PRSs,
applying the polygenic risk model to corresponding unadmixed genotype segments of an unadmixed ancestry reference cohort corresponding to the same ancestry label as the ancestry specific grouping to generate one or more unadmixed ancestry raw partial PRSs,
determining a mean PRS and a standard deviation PRS for the unadmixed ancestry reference cohort based on the one or more unadmixed ancestry raw partial PRSs,
normalizing the one or more ancestry specific raw partial PRSs based on the mean PRS and standard deviation PRS to generate normalized partial PRSs, and
generating the admixed PRS for the admixed subject based on a weighted sum of the normalized partial PRSs for each ancestry specific grouping.

17. The method of claim 16, further comprising: classifying the admixed individual as having an elevated risk for a particular disease based on the admixed PRS.

18. The method of claim 16, wherein normalizing the one or more ancestry specific raw partial PRSs further comprises:
centering the one or more ancestry specific raw PRSs based on the mean PRS and standard deviation PRS; and
scaling the one or more ancestry specific raw PRSs based on the mean PRS and standard deviation PRS.

19. The method of claim 16, further comprising obtaining the one or more phased subject genotype segments, including:
obtaining a biological sample from the admixed subject;
based on a DNA sample of the biological sample, performing genome sequencing and genotyping to determine an admixed genotype of the subject; and
phasing the admixed genotype of the subject to obtain the one or more phased subject genotype segments.

20. The method of claim 16, wherein:
a phased subject genotype segment is a marker or haplotype, and
assigning the ancestry label to the one or more phased subject genotype segments further comprises at least one of:
assigning the ancestry label to each marker of a phased subject genotype based on a reference cohort of known ancestry samples; and
assigning each haplotype of the phased subject genotype the ancestry label.

21. The method of claim 16, further comprising:
obtaining an admixed genotype from the admixed subject; and
phasing the subject genotype to generate the one or more phased subject genotype segments.

22. The method of claim 21, wherein phasing of the admixed genotype is performed using one or more of population-based methods or molecular based methods.

23. The method of claim 21, further comprising:
performing whole genome sequencing on a biological sample obtained from the admixed subject to determine the admixed genotype.

24. The method of claim 16, wherein generating the admixed PRS further comprises:
determining an ancestry specific summation for each ancestry specific grouping based on the corresponding normalized partial PRSs and a global ancestry fraction; and
determining the admixed PRS based on each ancestry specific summation for the one or more ancestry specific groupings.

25. The method of claim 16, wherein generating the admixed PRS further comprises:
determining an ancestry specific summation for each ancestry specific grouping based on the corresponding normalized partial PRSs, a global ancestry fraction, and a full PRS model effect size parameter; and
determining the admixed PRS based on each ancestry specific summation for the one or more ancestry specific groupings.

26. The method of claim 16, wherein generating the admixed PRS further comprises:
determining an ancestry specific summation for each ancestry specific grouping based on the corresponding normalized partial PRSs, a global ancestry fraction, and a partial PRS model effect size parameter; and
determining the admixed PRS based on each ancestry specific summation for the one or more ancestry specific groupings.

27. The method of claim 16, further comprising:
identifying one or more unadmixed ancestry training sets which correspond to each unadmixed ancestry cohort, wherein each unadmixed ancestry training set comprises one or more unadmixed ancestry training genotype segments; and
for each unadmixed ancestry training set:
applying a polygenic risk model corresponding to the ancestry label of the unadmixed ancestry training set to each unadmixed ancestry training genotype segment of the unadmixed ancestry training set to generate one or more unadmixed ancestry training partial PRSs, and
normalizing the one or more unadmixed ancestry training partial PRSs based on the mean PRS and standard deviation PRS to generate normalized unadmixed ancestry training partial PRSs.

28. The method of claim 27, further comprising:
determining a partial PRS model effect size parameter using a regression model based on each unadmixed ancestry training partial PRSs.

29. The method of claim 16, further comprising:
identifying one or more unadmixed ancestry training sets which correspond to each unadmixed ancestry cohort, wherein each unadmixed ancestry training set comprises one or more unadmixed ancestry training genotype segments and each unadmixed ancestry training genotype segment corresponds to a complete genotype of a corresponding unadmixed individual; and
for each unadmixed ancestry training set:
applying a polygenic risk model corresponding to the ancestry label of the unadmixed ancestry training set to each unadmixed ancestry training genotype segment of the unadmixed ancestry training set to generate one or more unadmixed ancestry training full PRSs, and
normalizing the one or more unadmixed ancestry training full PRSs based on the mean PRS and standard deviation PRS to generate normalized unadmixed ancestry training full PRSs.

30. The method of claim 29, further comprising:
determining a full PRS model effect size parameter using a regression model based on each unadmixed ancestry training full PRSs.

31. A system for generating an admixed polygenic risk score (PRS) for an admixed subject, the system comprising:
a processor; and
a memory storing software instructions that, when executed by the processor, cause the apparatus to perform steps including:
assigning an ancestry label to one or more phased subject genotype segments;
generating one or more ancestry specific groupings, wherein each ancestry specific grouping comprises the one or more phased subject genotype segments corresponding to a particular ancestry label;
for each ancestry specific grouping
applying a polygenic risk model corresponding to the ancestry label of the ancestry specific grouping to each phased subject genotype segment of the ancestry specific grouping to generate one or more ancestry specific raw partial PRSs,
applying the polygenic risk model to corresponding unadmixed genotype segments of an unadmixed ancestry reference cohort corresponding to the same ancestry label as the ancestry specific grouping to generate one or more unadmixed ancestry raw partial PRSs,
determining a mean PRS and a standard deviation PRS for the unadmixed ancestry reference cohort based on the one or more unadmixed ancestry raw partial PRSs, and
normalizing the one or more ancestry specific raw partial PRSs based on the mean PRS and standard deviation PRS to generate normalized partial PRSs; and
generating the admixed PRS for the admixed subject based on a weighted sum of the normalized partial PRSs for each ancestry specific grouping.

32. The system of claim 31, wherein the steps further include: classifying the admixed individual as having an elevated risk for a particular disease based on the admixed PRS.

33. The system of claim 31, wherein normalizing the one or more ancestry specific raw partial PRSs further comprises:
centering the one or more ancestry specific raw PRSs based on the mean PRS and standard deviation PRS; and
scaling the one or more ancestry specific raw PRSs based on the mean PRS and standard deviation PRS.

34. A computer program product for generating an admixed polygenic risk score (PRS) for an admixed subject, the computer program product comprising at least one non-transitory computer-readable storage medium storing software instructions that, when executed by an apparatus, cause the apparatus to perform steps including:
assigning an ancestry label to one or more phased subject genotype segments;
generating one or more ancestry specific groupings, wherein each ancestry specific grouping comprises the one or more phased subject genotype segments corresponding to a particular ancestry label;
for each ancestry specific grouping
applying a polygenic risk model corresponding to the ancestry label of the ancestry specific grouping to each phased subject genotype segment of the ancestry specific grouping to generate one or more ancestry specific raw partial PRSs,
applying the polygenic risk model to corresponding unadmixed genotype segments of an unadmixed ancestry reference cohort corresponding to the same ancestry label as the ancestry specific grouping to generate one or more unadmixed ancestry raw partial PRSs,
determining a mean PRS and a standard deviation PRS for the unadmixed ancestry reference cohort based on the one or more unadmixed ancestry raw partial PRSs, and
normalizing the one or more ancestry specific raw partial PRSs based on the mean PRS and standard deviation PRS to generate normalized partial PRSs; and
generating the admixed PRS for the admixed subject based on a weighted sum of the normalized partial PRSs for each ancestry specific grouping.

35. The computer program product of claim 34, wherein the steps further include: classifying the admixed individual as having an elevated risk for a particular disease based on the admixed PRS.
